(19) United States
(12) Patent Application Publication
(10) Pub. No.: US 2011/0207992 A1
(43) Pub. Date: Aug. 25, 2011

(54) IMPLANTABLE URETHRAL PROSTHESIS HAVING TABLE MEMBER

(76) Inventor: Allen F. Morey, Dallas, TX (US)

(21) Appl. No.: 13/030,736

(22) Filed: Feb. 18, 2011

Related U.S. Application Data
(60) Provisional application No. 61/305,634, filed on Feb. 18, 2010.

(51) Int. Cl. A61F 2/02

(52) U.S. Cl. 600/30

(57) ABSTRACT

An implantable urethral prosthesis for the treatment of male incontinence and method of implementing the same includes a support portion, an inflatable member, a reservoir-pump and a restrictor. The support portion includes at least a first set of two arms extending from the support portion, each arm of the first set of two arms configured to be secured around a respective descending rami of a pelvis of a patient, and a second set of two arms extending from the support portion and configured to be secured into tissue anterior to the pelvis to operatively place the support portion under the patient's urethra. The inflatable member is attachable to the support portion and adapted to exert a force on the urethra when inflated with an inflating agent.

### CROSS-REFERENCE TO RELATED APPLICATIONS

This application claims benefit of U.S. Provisional Patent Application Ser. No. 61/305,634, filed on Feb. 18, 2010, entitled “Inflatable Sling for Male Incontinence,” the contents of which are incorporated in their entirety herein by reference.

### BACKGROUND

Implantable devices are available that provide support to anatomical organs of a patient to treat urinary incontinence. Such devices include sub-urethral slings that are surgically implanted under a patient's urethra to provide support to the urethra to inhibit urine from leaking out of the urethra during a provocative event such as coughing or laughing.

Implanting and anatomically securing some sling devices can be difficult and time consuming. In addition, in the case of urinary incontinence, some sling devices may provide unreliable anatomical fixation and/or imperfect tensioning for supporting the urethra, thereby leading to suboptimal or even unacceptable results for the treatment of urinary incontinence.

### SUMMARY

Embodiments provide an implant and methods of deploying an implant that are effective and easy to deploy in treating male incontinence. One embodiment of the implant in the form of a sling or a support that provides the combination of a urethral support portion, four anchoring portions and an inflatable member adjacent to the support portion. The support portion is operatively positioned at or distal to the bulbar urethra. The support portion is operative to provide structural support to the urethra such that during the occurrence of a provocative event, the sling is operative to compressively engage the urethra to prevent urine leaking there from. The support portion will have a surface area that is at least 2 cm long by 1 cm wide to 8 cm long by 8 cm wide. A reservoir-pump is in fluid communication with the inflatable member and is adapted to receive an inflating agent from, and transfer inflating agent to, the inflatable member.

The anchor portions extend from the support portion. The anchor portions, which may take a variety of configurations, are operative to extend through the obturator foramen of the patient and become secured at a locus preferably at or near the descending ramus or supra pubic region. To effectuate such attachment, it is contemplated that one or more sutures may be utilized to ensure that the anchor portions become secured about the ramus or supra pubic fascia and that the support portion remains operatively positioned at or distal to the bulbar urethra. In one embodiment, the implant will have at least two anchor portions extending from the support portion with each respective anchor portion extending through the obturator foramen and attachable about respective ones of the patient's rami. The anchor portions will have a width ranging from between 0.5-4 cm and the length ranging from at least 5.0 cm to 45.0 cm. The support and anchor portions of the implant may be surgically positioned by the surgeon to optimize sizing and configuration of the implant to suit the specific patient.

In certain embodiments of the implant, a central support structure with anchoring portions extending there from is fashioned to have generally “H”, inverted “U”, or “X” shapes. The implants may further be fabricated such that sutures are pre-attached thereto, per the teachings of Assignee's co-owned pending U.S. patent application Ser. No. 10/947,182, entitled READILY IMPLANTABLE SLING, filed Sep. 22, 2004, the teachings of which are expressly incorporated in entirety into this document by reference. The implants are suitably fabricated from synthetic materials, such as synthetic mesh and the like, or natural materials, such as harvested tissues from sources such as animals, cadavers or the patient himself. The implants may further be fabricated such that the same are formed from combinations of materials, including but not limited to combinations of different types of synthetic materials, combinations of different types of natural tissues and/or combinations of both synthetic and natural tissues.

In one embodiment the inflatable member is provided as an inflatable pillow that associates with the support portion and is positionable adjacent to or against the urethra. The inflatable member is adapted to selectively exert a force on the patient's urethra to prevent leakage from the bladder. A reservoir-pump is attached to the inflatable member and contains an inflating agent. A restrictor is included with the reservoir-pump such that the inflating agent can be transferred to and from the reservoir and the inflatable member, thereby achieving release or closure of the urethra proximate to the support portion and inflatable member of the implant.

Regardless of the embodiment, in all procedures involving the implantation of the implants of the present invention, such procedures are performed with the patient assuming a lithotomy position. A vertical perineal incision is made in the midline dissecting to expose the bulbar urethra and the inferior aspect of the descending rami bilaterally. A surgical introducer is utilized to introduce the implant into position, typically through the obturator foramen, in a manner that is substantially easier and less traumatic than conventional surgical procedures utilized to access and implant slings for the treatment of male incontinence. Once the surgical site is accessed, two of the anchoring portions will preferably be positioned through the obturator foramen and anchored into position about the descending rami and two anchoring portions will be positioned in supra pubic fascia. Surgical sutures and the like may be deployed to facilitate anchoring of the anchor portions of the implant. The inflatable member can be positioned adjacent to or attached directly to the support portion. Minor variations can be made to effectuate optimal attachment and positioning of the support portion of the implant, as may be necessary for a given patient. In all cases, however, the surgical procedures and implants of the present invention provide a radically more efficient, effective and less traumatic surgical approach to the treatment of male incontinence.

### BRIEF DESCRIPTION OF THE DRAWINGS

These and other features and advantages of the various embodiments disclosed herein will be better understood with respect to the following description and drawings, in which like numbers refer to like parts throughout.

FIG. 1A is a frontal perspective view of one embodiment of an implant implanted within a pelvis.

FIG. 1B is a frontal perspective view of another embodiment of an implant implanted within a pelvis.

FIG. 2 is a frontal view of one embodiment of an implant.

FIG. 3 is a view of one embodiment of an implant illustrating an inflatable member, a reservoir-pump, and a restrictor.

### DETAILED DESCRIPTION

In the specification and in the claims, the terms “including” and “comprising” are open-ended terms and should be interpreted to mean “including, but not limited to . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The above description is given by way of example, and not limitation. Given the above disclosure, one skilled in the art could devise variations that are within the scope and spirit of the invention disclosed herein, including various ways of making and surgically implanting the implants described herein. Further, the various features of the embodiments disclosed herein can be used alone, or in varying combinations with each other and are not intended to be limited to the specific combination described herein. Thus, the scope of the claims is not to be limited by the illustrated embodiments.

Embodiments provide a urethral prosthesis including a support portion, an inflatable member adjacent to support portion, a reservoir in fluid communication with the inflatable member and a restrictor. Upon compression of the inflatable member, an inflating agent is transferred from the inflatable member to a reservoir. Upon compression of the reservoir, the inflating agent returns to the inflatable member. The urethral prosthesis is implantable such that the inflatable member prevents unintentional voiding of a patient's bladder.

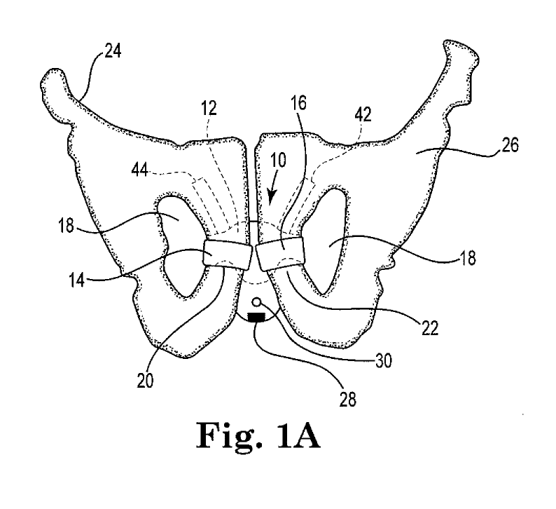

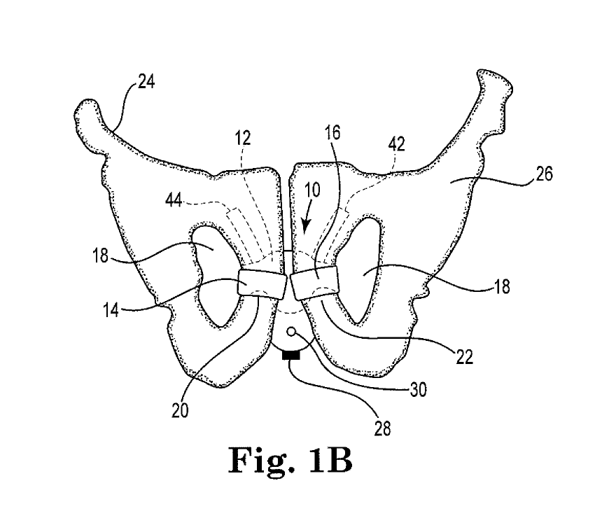

Referring now to the drawings, initially to FIGS. 1A and 1B, there are shown an implant 10 for the treatment of male incontinence that is operative to provide suburethral support to thus prevent the leakage of urine, particularly when the patient experiences a provocative event. In this respect, the implant 10 is operative to act as a suburethral sling but is advantageously operative to be more easily secured into position than prior art sling implants. The implants of the present invention are further capable of being deployed in a manner that is far less traumatic than prior art sling implants and methods of surgically implanting the same, and further utilizing a novel attachment approach that provides for optimal suburethral positioning of the sling 10 in an exceptionally secure manner without the use of bone anchors.

Embodiments provide a combination of a urethral support portion 12 and four(4) anchoring portions 14, 16, 42, 44. The support portion 12 is operatively positioned at or distal to the bulbar urethra that surrounds the urethra 30. The anchoring portions 14, 16, 42, 44 are operative to secure the support portion 12 into position to thus provide structural support to the urethra 30, and particularly so during the occurrence of a provocative event. To achieve that end, the anchoring members 14, 16, 42, 44 are generally operative to extend through the obturator foramen. Two of the anchoring members 14, 16 become secured at both of the patient's rami. Two of the anchoring members 42, 44 become secured within the patient's supra pubic fascia. In one embodiment discussed more fully below two of the anchor portions will be operatively interconnectable with the descending ramus of one of the patient's pelvis. Advantageously, the positioning of the anchor portion against the descending ramus will be accomplished without the use of bone screws, which can be problematic to deploy and utilized as an attachment point for sutures and the like.

Embodiments provide an implantable urethral prosthesis comprising an elongate strip of flexible material including first, second, third and fourth ends, all four ends adapted to be secured to soft tissue in the vicinity of a patient's descending rami and supporting the patient's urethra. The four arms of the elongate strip all protrude away from each other and away from a support portion of the sling. Such a construction is found in U.S. Pat. No. 7,431,690, the contents of which are incorporated herein by reference.

The 4 armed elongate strip of flexible material including first, second, third and fourth ends of anchoring members 14, 16, 42, 44 and the support portion 12 is also referred to as a urethral sling. That is, the urethral sling includes four arms 14, 16, 42, 44 that are useful for securing and positioning the sling within the body cavity and a support portion that is useful for supporting the urethra. Suitable previously known urethral slings and configurations include those disclosed, for example, in U.S. Pat. Nos. 7,621,864, 7,611,454, 7,431,690 and 7,422,557 and U.S. Patent Publication Nos. 2005/0283040, 2006/0052800 and 2006/0122457, the contents of which are included in their entirety. In one embodiment, the 4 armed urethral sling with a support portion is described in U.S. Pat. No. 7,431,690, the contents of which are incorporated in by reference in their entirety.

For purposes of defining the general parameters of implants of the present invention, it is presently believed that the support portion of the implant will define a surface area ranging from a 1 cm width by 2 cm length to an 8 cm width by 8.0 cm length. With respect to the anchor portion, the same will preferably define a surface area defined by a 0.5 cm width by 5.0 cm length to an area defined by a 4.0 cm width to a 45.0 cm length. With respect to the latter, it should further be understood that certain embodiments of the present invention, discussed more fully below, sutures may be utilized in operative combination with the anchoring portion or, in certain embodiments, exclusively with the support portion, to thus define means for attaching and securing the implant into position.

An inflatable member 28 is positioned adjacent to the 4 armed elongate strip, supporting the support portion 12. The inflatable member 28 is positioned about the urethra 30 and is adapted to exert a force on the patient's urethra 30 and 4 armed elongate strip when inflated with an inflating agent. A reservoir is in fluid communication with the inflatable member that is adapted to receive an inflating agent from, and transfer inflating agent to, the inflatable member. A restrictor can be adapted to regulate the transfer of the inflating agent between the reservoir and the inflatable member and wherein upon compression of the inflatable member, the inflating agent transfers from the inflatable member to the reservoir/pump (a “resi-pump”; a suitable pump that has both storage capability and pumping capability).

The implants are suitably fabricated from either synthetic materials, such as surgical mesh and the like, natural tissues, such as tissues harvested from either an animal, cadaverous source or the patient himself, and/or combinations of synthetic and natural materials. Along these lines, it is contemplated that the implants of the present invention can be fabricated consistent with those methods disclosed in Assignee's co-owned and co-pending U.S. patent application Ser. No. 10/684,861, the teachings of which are expressly incorporated herein by reference. Along these lines, it is further contemplated that in certain embodiments of the present invention, the implants may be prefabricated with slings or sutures attached thereto, consistent with the teachings of Applicants' co-owned pending U.S. patent application Ser. No. 10/947,182, entitled READILY IMPLANTABLE SLING, the teachings of which are likewise incorporated by reference.

Inflatable member is suitably fabricated from polymers such as polyethylene, polypropylene, polyurethanes at the like.

Suitable inflating agents include gases and liquids, for example, aqueous solutions, such as saline or sterile water.

Suitable restrictors, such as ball valves or check valves, are adapted to regulate the flow of inflating agent between the inflatable member and the reservoir and is not meant to be limiting. More particularly, the restrictor allows inflating agent to flow from the reservoir to the inflatable member during compression of the reservoir, but resists flow of inflating agent from the inflatable member to the reservoir.

The length of the tubes can be varied depending on the anatomy of the patient. In one aspect, it is possible to secured the reservoir-pump within the scrotum of the patient. Otherwise, it is possible to have the reservoir-pump system remain attached to the inflatable member and protrude from the patients soft tissue.

Soft tissue includes dermal tissue, sub-dermal tissue, ligaments, tendons, or membranes but does not generally include bone.

Embodiments provide a device configured to selectively coapt a tubular member (the urethra) of a human body (male or female) in a manner that reduces or eliminates erosion of the tubular member.

Embodiments provide a device configured to hydraulically occlude and thus impede liquid flow through a urethra.

The patient selectively inflates the member to close the urethra to prevent leakage of urine. Subsequently, the patient selectively deflates the member to release the pressure on the urethra to open a pathway for the passage of urine. The pump can be implanted in the scrotum.

Referring again to FIGS. 1 and 1B, implant 10 is situated such that the support portion 12 thereof is operatively positioned over the urethra 30. The implant 10 as depicted includes first and second anchor portions 14, 16 that are shown extending through the obturator foramen 18 and wrapped about dedicated ones of the descending rami 20, 22 of hip bones 24, 26, respectively. Anchor portions 42 and 44 are shown by broken lines and are positioned and secured within the patient's supra pubic fascia. The surgeon determines what positions provide the most beneficial support.

In FIG. 1A, inflatable member 28 is located between support portion 12 and urethra 30. In FIG. 1B, support portion 12 is located between inflatable member 28 and urethra 30.

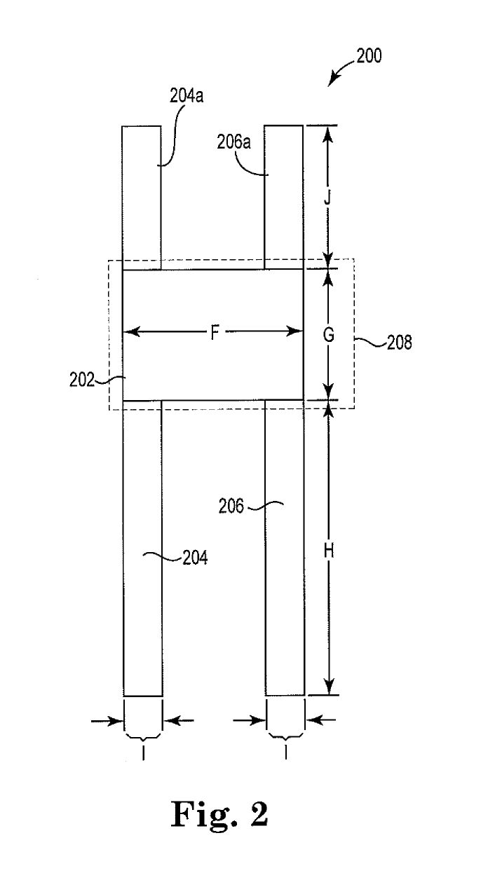

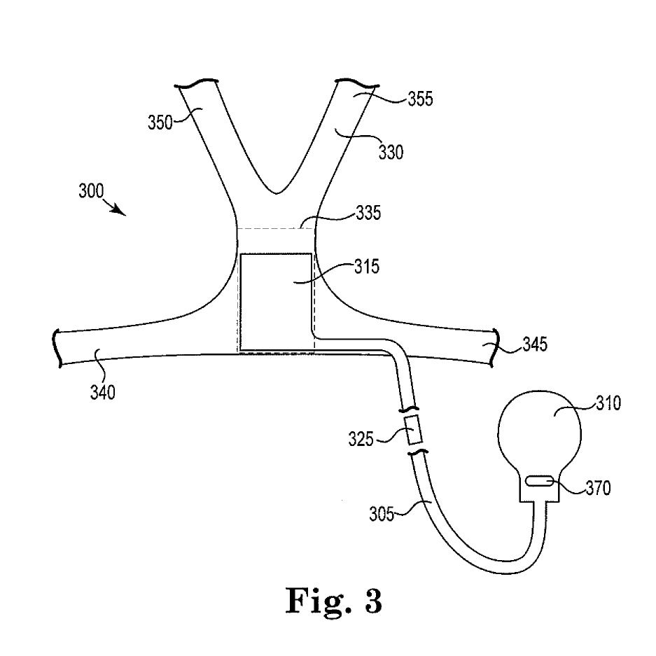

FIGS. 2 and 3 illustrate various embodiments of the implants of the present invention. Referring initially to FIG. 2, there is shown an embodiment 200 for use in the surgical treatment of male urinary incontinence. As depicted, the implant 200 includes a support portion 202 with first and second anchor portions 204, 206 depending there from in generally parallel relation to one another, additional anchor portions 204a, 206a, ascending there from to define an “H” shape and an inflatable member 208 (broken lines). The implant 200, as depicted, is preferably formed such that support portion 202 has a width “F” of approximately 7 cm and a height “G” of approximately 5 cm. Anchor portions 204, 206 preferably have a length “H” of approximately 30 cm and a width “I” of 1 cm. Additional anchor portions 204a, 206a will have a width of approximately 1.5 cm and a height “J” of approximately 14 cm, and may preferably be formed as extensions of 204 and 206, respectively. Again, such dimensions can be varied as may be deemed appropriate by one skilled in the art.

In the embodiment depicted, anchor portions 204, 206 are operative to extend through the retropubic space, from posterior to symphysis, or may extend via a transobturator route. Anchor portions 204a, 206a are operative to pass through the rectus fascia from anterior to symphysis.

The surgical procedure for implanting implant 200 can be conducted with the patient assuming a lithotomy position whereby a vertical perineal incision will be made in the midline dissecting to expose the bulbar urethra in the descending pubic rami bilaterally. The bulbar spongiosis will be left intact. Thereafter, suprapubic transverse incisions of approximately 1 cm are made, 3 cm lateral to the midline on either side. A suitable introducer is then passed from such incisions and tunneled superficially anterior to the pubic symphysis (not retropubically) and caused to exit through the initial perineal incision. A respective one of the ascending anchor portions 204a or 206a is attached to a suprapubic introducer and pulled through the suprapubic incisions. This same maneuver is then repeated for the respective other ascending anchor portion 204a or 206a.

Hereafter, two new incisions, which are preferably approximately 1 cm above the pubic symphysis are performed. The suitable introducer is then passed from above, retropubically, and out through the initial perineal incision. A respective one of the descending anchor portions 204 or 206 is then attached to the introducer and pulled up through such newly formed incision. Such procedure is repeated with respect to the other descending anchor portion 204 or 206, which as a consequence creates two sets of anchor portions, namely, descending anchor portions 204, 206 and ascending portions 204a, 206a in the suprapubic region. The ascending anchor portions 404a, 406a are then tunneled into the retropubic anchor portions 204, 206 beneath the skin and thereafter ascending portion 204a will be surgically connected with descending portion 204 and ascending portion 206a surgically attached to descending anchor portion 206. To achieve such surgical attachment, it is contemplated that such attachment may be attained via the use of sutures.

In a further refinement of such procedure, it is contemplated that surgical positioning of descending anchor portions 204, 206 can be accomplished via the use of a suitable introducer that is passed from outside in or inside out through the obturator foramen. A suture that is attached to the distal most end of respective one of anchor portions 204, 206 will be threaded to the introducer. Once the introducer is advanced through the obturator foramen, the same is retracted with the suture affixed to the end of descending anchor portion 204 or 206 is pulled until the end of the anchor portion 204 or 206 can be grasped. A second suitable introducer is utilized with respect to the surgical positioning of the respective other anchor portion 204 or 206 on the contra-lateral side. Once so positioned, anchor portions 204 and 206 can be affixed to ascending anchor portions 204a, 206a around the descending rami via the transobturator approach and secured on itself via a suture line.

FIG. 3 is a perspective view of a device 300 according to one embodiment. Device 300 includes a reservoir-pump 310, a 4 armed support 330 coupled to an inflatable member 315, a conduit 320 (and 305) providing fluid communication between reservoir-pump 310 and inflatable member 315, an optional connector 325 and restrictor 370.

In one embodiment, 4 armed support 330 includes first, second, third and fourth connection ends 350, 355, 340 and 345, where the ends are each attachable to soft tissue in a patient to position inflatable member 315 relative to the urethra of the patient. Support 330 also includes a support portion, 335 (noted in broken lines of FIG. 3); from which connection ends 350, 355, 340 and 345 protrude from. Support portion 315 is configured to approximate the size of inflatable member 315. Thus support portion 335 and inflatable member 315 are positioned against or in relation to the urethra so as to constrict the urethra upon filling of the inflatable member 315. Reservoir-pump 310 couples with and is configured to selectively inflate inflatable member 315. In this manner, inflatable member 315 is anchored relative to the urethra, and inflation of inflatable member 315 compresses the urethra to impede liquid flow through the urethra.

In one embodiment, reservoir-pump 310 includes a reservoir-pump conduit 305 that is removably attachable to an inflatable member conduit 320, for example via a connector 325. In one embodiment, reservoir-pump conduit 305 is integrally formed as a single piece with conduit 320, such that connector 325 is not present.

In one embodiment, 4 armed support 330 with support portion 335 and inflatable member 315 are integrally molded as a single unit. Inflatable member 315 may be fabricated in a rectilinear form or a curvilinear form or in other suitable forms. For example, in one embodiment inflatable member 315 is molded to provide a pillow to be disposed in a complimentary fashion relative to the bulbous spongiosum surrounding the urethra.

In one embodiment, inflatable member 315 is provided with a length between about 2 and about 5 cm and having a width between about 3 and about 5 cm, although the size of inflatable member 315 is not critical. In similar fashion, support portion 335 has a length between about 2 and about 5 cm and having a width between about 3 and about 5 cm.

In one embodiment, reservoir-pump 310 is formed of silicone and includes an articulated housing that is configured to be sufficiently resilient to enable a patient to expel a sufficient volume of liquid from reservoir-pump 310 to inflate inflatable member 315. In one embodiment, reservoir-pump 310 is sufficiently resilient to enable the patient to expel a sufficient volume of liquid from reservoir-pump 310 to inflate inflatable member 315 with one squeeze. Other styles and forms of squeezable bulbs/pumps are also acceptable. Suitable liquids to be retained in reservoir-pump 310 for activating inflatable member 315 include fluids in general, examples of which include water or a saline solution of water. Preferably the liquid is a sterile saline solution.

In one embodiment, reservoir-pump 310 encloses restrictor 370 which can be a ball valve or check valve 380, a biasing member 382 (not shown), and a seat 384 (not shown) sized to receive restrictor 370. In one embodiment, compressing reservoir-pump 310 ejects the liquid volume V into or toward reservoir-pump conduit 305 with sufficient force to lift ball valve 380 off of seat 384. Biasing member 382 is subsequently compressed, which provided a fluid passageway between ball valve 380 and seat 384 that allows the liquid inside of reservoir-pump 310 to flow through reservoir-pump conduit 305 and inflate inflatable pillow 315. When the pressure (e.g., squeeze) applied to reservoir-pump 310 is relieved, biasing member 382 biases ball valve 380 back into engagement with seat 384, which closes the fluid passageway between ball valve 380 and seat 384 to ensure that the liquid remains in inflatable member 315 and inflatable member 315 remains inflated. In this state, reservoir-pump 310 is “cavitated” to have a lower pressure than the pressure in inflatable member 315 back into reservoir-pump 310 when ball valve 380 is displaced from seat 384, for example when the patient desires to deflate inflatable member 315.

In a particular embodiment (not shown), restrictor 370 can be positioned between reservoir-pump 310 and conduit 305 utilizing TruLock™ connector technology, presently used in the Titan™ inflatable penile prostheses, and described in U.S. Pat. No. 4,890,866, entitled “Tubing Connector”. This can be used to connect the reservoir-pump 310 tubing to conduit 305 connecting to inflatable member 315.

In one embodiment, and with reference to FIG. 3, conduit 305 between reservoir-pump 310 and inflatable member 315 is provided in a fixed length such that reservoir-pump conduit 305 and inflatable member conduit 320 are formed as a single integral conduit. However, it may be desirable (e.g., for differently sized patients) to provide a connector 325 to couple reservoir-pump conduit 305 to an inflatable member conduit 315 of a selected length to accommodate patients ranging in size from adolescent to adult sized.

One example of a surgical method to implant device 300 for treatment of urinary incontinence in a patient follows. Although the following procedure is described in relation to male anatomy in which the device is employed to support the male urethra, one of ordinary skill in the art will realize that similar such procedures relating to implanting embodiments of the device into female anatomy are also possible. For example, in one embodiment device 300 is implanted in a female patient to support the female urethra with substantially zero tension applied to the urethra. In one embodiment, device 300 is implanted in a female patient to support the female urethra with a non-zero tension applied to the urethra.

A catheter is placed in the patient's urethra U, among other usual and preliminary steps in preparation for surgery. The patient is placed on an operating table in a slightly exaggerated lithotomy position with buttocks extending just beyond an edge of the table. With the patient under anesthesia, a vaginal incision (female) or a perineal incision (male) is made followed by blunt dissection. The surgeon will utilize the procedure for affixing the 4 armed urethral sling as generally described in U.S. Pat. No. 7,431,690, the contents of which are incorporated herein in their entirety.

As depicted, implant 300 includes a support portion 335 with first and second anchor portions 340, 345 depending there from in generally parallel relation to one another and additional anchor portions 350, 355, ascending there from to define an “H” shape. Implant 300, as depicted, is preferably formed such that support portion 335 has a width of approximately 3 cm and a height of approximately 2 cm. Anchor portions 340, 345 preferably have a length of approximately 30 cm and a width of 1 cm. Additional anchor portions 350, 355 have a width of approximately 1.5 cm and a length of approximately 14 cm. Such dimensions can be varied as may be deemed appropriate by one skilled in the art.

In the embodiment depicted in FIG. 3, anchor portions 340, 345 are operative to extend through the retropubic space, from posterior to symphysis, or may extend via a transobturator route. Anchor portions 350, 355 are operative to pass through the rectus fascia from anterior to symphysis.

The surgical procedure for implanting implant 300 can be conducted with the patient assuming a lithotomy position whereby a vertical perineal incision will be made in the midline dissecting to expose the bulbar urethra in the descending pubic rami bilaterally. The bulbar spongiosis will be left intact. Thereafter, suprapubic transverse incisions of approximately 1 cm are made, 3 cm lateral to the midline on either side. A suitable introducer is then passed from such incisions and tunneled superficially anterior to the pubic symphysis (not retropubically) and caused to exit through the initial perineal incision. A respective one of the ascending anchor portions 350 or 355 is attached to a suprapubic introducer and pulled through the suprapubic incisions. This same maneuver is then repeated for the respective other ascending anchor portion 350 or 355.

Thereafter, two new incisions, which are preferably approximately 1 cm above the pubic symphysis, are performed. A suitable introducer is then passed from above, retropubically, and out through the initial perineal incision. A respective one of the descending anchor portions 340 or 345 is then attached to the introducer and pulled up through such newly formed incision. Such procedure is repeated with respect to the other descending anchor portion 340 or 345, which as a consequence creates two sets of anchor portions, namely, descending anchor portions 340, 345 and ascending portions 350, 355 in the suprapubic region. The ascending anchor portions 350, 355 are then tunneled into the retropubic anchor portions 340, 345 beneath the skin and thereafter ascending portion 350 will be surgically connected with descending portion 340 and ascending portion 355 surgically attached to descending anchor portion 345. To achieve such surgical attachment, it is contemplated that such attachment may be attained via the use of sutures.

In a further refinement of such procedure, it is contemplated that surgical positioning of descending anchor portions 340, 345 can be accomplished via the use of a suitable introducer that is passed from outside in or inside out through the obturator foramen. A suture that is attached to the distal most end of respective one of anchor portions 340, 345 can be threaded to the introducer. Once the introducer is advanced through the obturator foramen, the same is retracted with the suture affixed to the end of descending anchor portion 340 or 345 is pulled until the end of the anchor portion 340 or 345 can be grasped. A second suitable introducer is utilized with respect to the surgical positioning of the respective other anchor portion 340 or 345 on the contra-lateral side. Once so positioned, anchor portions 340 and 345 can be affixed to ascending anchor portions 350, 355 around the descending rami via the transobturator approach and secured on itself via a suture line.

It should be understood that inflatable member 315 would be situated about the support portion 335 and the urethra such that inflation and deflation of inflatable member 315 will constrict the urethra and passage of urine. The surgeon will determine positioning of inflatable member 315 with respect to support position and anatomy of the individual patient. Inflatable member 315 can be attached to the undersurface of support portion 335. For example, it can be fused to the sling 330 as a part of manufacturing or sewn to the sling by leaving a small opening in each of its 4 corners to anchor it into place with sutures at its periphery. It should be understood that inflatable member 315 can include a surface larger than support portion 335, smaller than the surface of support portion 335 or of the same size as support portion 335.

Device 300 is preferably placed with inflatable member 315 deflated. With device 300 thus placed and secured in the patient by way of fixed anchor and adjustable anchor, an assessment is made of whether support 330 is unacceptably loose or tight under the urethra. If support 330 is unacceptably loose or unacceptably tight, adjustments can be made. The incision is subsequently closed and usual post-operative procedures are performed. After a suitable surgeon-determined time for healing, a volume of liquid is post-surgically injected into reservoir-pump 310 and inflatable member 315 is inflated sufficiently to occlude the urethra. The surgeon may choose to adjust the volume of liquid in reservoir-pump 310 at this stage to control the rate and pressure of the inflation of inflatable member 315.

With regard to suitable placement of reservoir-pump 310, the groin area of the patient can be shaved, cleaned and suitably prepped with a surgical solution prior to draping with a sterile drape. Some surgeons place a retraction device, such as a retractor sold under the trademark Lone Star and available from Lone Star Medical Products of Stafford, Tex., around the penis. Thereafter, the surgeon forms a transverse scrotal incision (across an upper portion of the patient's scrotum) to access the scrotum for placement of the reservoir-pump. As an example of the transverse scrotal approach, the surgeon forms a 2-3 cm transverse incision through the subcutaneous tissue of the median raphe of the upper scrotum to form an opening in the subdartos space that is sized to receive the reservoir-pump. Alternatively, the reservoir-pump can be advanced into a subdartos pocket within the scrotum created by dissecting up from the top of the perineal incision.

Device 300 enables the surgeon to selectively adjust the location of support 330 relative to the urethra. The distance between the obturator foramen OF and the urethra will vary by individual, and device 300 desirably provides the surgeon with adjustability to accommodate the different sizes of these individuals.

The following paragraphs enumerated consecutively from 1 through 14 provide for various aspects of the present invention. In one embodiment, in a first paragraph (1), the present invention provides an implantable urethral prosthesis comprising an implantable urethral prosthesis comprising:

a support portion including at least a first set of two arms extending from the support portion, each arm of the first set of two arms configured to be secured around a respective descending rami of a pelvis of a patient, and a second set of two arms extending from the support portion and configured to be secured into tissue anterior to the pelvis to operatively place the support portion under the patient's urethra;

an inflatable member attachable to the support portion and adapted to exert a force on the urethra when inflated with an inflating agent;

a reservoir-pump in fluid communication with the inflatable member that is adapted to receive an inflating agent from, and transfer inflating agent to, the inflatable member; and

a restrictor adapted to regulate the transfer of the inflating agent between the reservoir-pump and the inflatable member and wherein upon compression of the reservoir, the inflating agent transfers from the reservoir to the inflatable member.

2. The urethral prosthesis of paragraph 1 wherein the support portion comprises a first surface configured to be placed adjacent to the urethra and a second surface opposite the first surface, inflatable member attachable to the first surface of the support portion.

3. The urethral prosthesis of paragraph 1 wherein the support portion comprises a first surface configured to be placed adjacent to the urethra and a second surface opposite the first surface, inflatable member attachable to the second surface of the support portion.

4. The urethral prosthesis of paragraph 1 wherein the support portion and the inflatable member are integrally molded as a single unit.

5. The urethral prosthesis of paragraph 1 wherein the support portion comprises a mesh material.

6. The urethral prosthesis of paragraph 1 wherein the inflatable member is sized to approximate the dimensions of a central region of the support portion.

7. A method of surgically implanting the urethral prosthesis of paragraph 1 into a patient, the method comprising:
creating a perineal incision;
creating suprapubic incisions;
inserting the urethral support portion including the inflatable member into the patient through the perineal incision;
passing an introducer from the suprapubic incisions, tunneling the introducer superficially anterior to the pubic symphysis, and exiting the introducer through the initial perineal incision;
creating incisions above the pubic symphysis;
passing an introducer from above, retropubically, and out through the initial perineal incision;
passing an arm up through the pubic symphysis incision and tunneling ascending anchor portions into the retropubic region beneath the skin.

8. The method of paragraph 7, further comprising inflating the inflatable member and evaluating tension of the support portion applied to the urethra.

9. The method of paragraph 8, further comprising adding an additional volume of an inflation agent to the inflatable member and increasing the tension of the support portion applied to the urethra.

10. The method of paragraph 9, further comprising removing a portion of a volume of an inflation agent from the inflatable member and loosening the tension of the support portion applied to the urethra.

11. A method of treating urinary incontinence comprising:
creating a perineal incision;
inserting the urethral support portion of paragraph 1 including the inflatable member into the patient through the perineal incision;
securing the urethral support portion between two opposing obturator membranes and two suprapubic fascia sites such that the inflatable bladder is spaced apart from the urethra; and
evaluating the tension of the support portion relative to the urethra.

12. The method of paragraph 11, further comprising inflating the inflatable member and coapting the urethra.

13. A kit of pieces comprising:
a support portion including at least a first set of two arms extending from the support portion, each arm of the first set of two arms configured to be secured around a respective descending rami of a pelvis of a patient, and a second set of two arms extending from the support portion and configured to be secured into tissue anterior to the pelvis to operatively place the support portion under the patient's urethra;

an inflatable member attachable to the support portion and adapted to exert a force on the urethra when inflated with an inflating agent;

a reservoir-pump in fluid communication with the inflatable member that is adapted to receive an inflating agent from, and transfer inflating agent to, the inflatable member; and

a restrictor adapted to regulate the transfer of the inflating agent between the reservoir-pump and the inflatable member.

14. The kit of paragraph 13, further comprising instructions for the implantation of the kit pieces.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention. Along these lines, the suggested surgical implantation techniques with respect to each of the depicted embodiments simply represent the Applicant's best mode for practicing the invention. In addition, it is contemplated that future advances in medicine can and will be made that will facilitate the ability of the implants of the present invention to be more easily, quickly and accurately secured into position and operative to achieve the highest degree of favorable patient outcome. In addition, it is emphasized again that although dimensions are provided herein with respect to the implants of the present invention, as well as the choice of materials that may be utilized as to fabricate the same, it should be understood that any suitable material useful for fabricating an implant should be deemed to fall within the scope of the present invention.

What is claimed is:

1. An implantable urethral prosthesis comprising:
a support portion including at least a first set of two arms extending from the support portion, each arm of the first set of two arms configured to be secured around a respective descending rami of a pelvis of a patient, and a second set of two arms extending from the support portion and configured to be secured into tissue anterior to the pelvis to operatively place the support portion under the patient's urethra;
an inflatable member attachable to the support portion and adapted to exert a force on the urethra when inflated with an inflating agent;
a reservoir-pump in fluid communication with the inflatable member that is adapted to receive an inflating agent from, and transfer inflating agent to, the inflatable member; and
a restrictor adapted to regulate the transfer of the inflating agent between the reservoir-pump and the inflatable member and wherein upon compression of the reservoir, the inflating agent transfers from the reservoir to the inflatable member.

2. The urethral prosthesis of claim 1 wherein the support portion comprises a first surface configured to be placed adjacent to the urethra and a second surface opposite the first surface, inflatable member attachable to the first surface of the support portion.

3. The urethral prosthesis of claim 1 wherein the support portion comprises a first surface configured to be placed adjacent to the urethra and a second surface opposite the first surface, inflatable member attachable to the second surface of the support portion.

4. The urethral prosthesis of claim 1 wherein the support portion and the inflatable member are integrally molded as a single unit.

5. The urethral prosthesis of claim 1 wherein the support portion comprises a mesh material.

6. The urethral prosthesis of claim 1 wherein the inflatable member is sized to approximate the dimensions of a central region of the support portion.

7. A method of surgically implanting the urethral prosthesis of claim 1 into a patient, the method comprising:
creating a perineal incision;
creating suprapubic incisions;
inserting the urethral support portion including the inflatable member into the patient through the perineal incision;
passing an introducer from the suprapubic incisions, tunneling the introducer superficially anterior to the pubic symphysis, and exiting the introducer through the initial perineal incision;
creating incisions above the pubic symphysis;
passing an introducer from above, retropubically, and out through the initial perineal incision;
passing an arm up through the pubic symphysis incision and tunneling ascending anchor portions into the retropubic region beneath the skin.

8. The method of claim 7, further comprising inflating the inflatable member and evaluating tension of the support portion applied to the urethra.

9. The method of claim 8, further comprising adding an additional volume of an inflation agent to the inflatable member and increasing the tension of the support portion applied to the urethra.

10. The method of claim 9, further comprising removing a portion of a volume of an inflation agent from the inflatable member and loosening the tension of the support portion applied to the urethra.

11. A method of treating urinary incontinence comprising:
creating a perineal incision;
inserting the urethral support portion of claim 1 including the inflatable member into the patient through the perineal incision;
securing the urethral support portion between two opposing obturator membranes and two suprapubic fascia sites such that the inflatable bladder is spaced apart from the urethra; and
evaluating the tension of the support portion relative to the urethra.

12. The method of claim 11, further comprising inflating the inflatable member and coapting the urethra.

13. A kit of pieces comprising:
a support portion including at least a first set of two arms extending from the support portion, each arm of the first set of two arms configured to be secured around a respective descending rami of a pelvis of a patient, and a second set of two arms extending from the support portion and configured to be secured into tissue anterior to the pelvis to operatively place the support portion under the patient's urethra;
an inflatable member attachable to the support portion and adapted to exert a force on the urethra when inflated with an inflating agent;
a reservoir-pump in fluid communication with the inflatable member that is adapted to receive an inflating agent from, and transfer inflating agent to, the inflatable member; and
a restrictor adapted to regulate the transfer of the inflating agent between the reservoir-pump and the inflatable member.

14. The kit of claim 13, further comprising instructions for the implantation of the kit pieces.
